(19) United States
(12) Patent Application Publication
(10) Pub. No.: US 2017/0340866 A1
(43) Pub. Date: Nov. 30, 2017

(54) TISSUE DILATORS

(71) Applicant: Covidien LP, Mansfield, MA (US)

(72) Inventor: Paul D. Richard, Shelton, CT (US)

(21) Appl. No.: 15/466,904

(22) Filed: Mar. 23, 2017

Related U.S. Application Data
(60) Provisional application No. 62/343,206, filed on May 31, 2016.

(51) Int. Cl. A61M 29/00; A61B 17/3207

(52) U.S. Cl. CPC A61M 29/00; A61B 17/320758; A61B 2017/320775

(57) ABSTRACT

A tissue dilator includes a plurality of segments pivotable between a first configuration to define a first channel, and a second configuration, to define a second channel, larger in size than the first channel.

### CROSS-REFERENCE TO RELATED APPLICATIONS

This application claims the benefit of and priority to U.S. Provisional Patent Application No. 62/343,206 filed May 31, 2016, the entire disclosure of which is incorporated by reference herein.

### BACKGROUND

### Technical Field

The present disclosure relates generally to tissue dilators used during surgical procedures or medical examinations. More particularly, the present disclosure relates to tissue dilators used in minimally invasive surgical procedures and/or methods of using the disclosed tissue dilators.

### Background of Related Art

During a minimally invasive surgical procedure or a medical examination, access to an interior portion of a patient may be required. For example, in a laparoscopic hysterectomy procedure, entry into the abdominal cavity is required to allow for the passage of surgical instruments and the ultimate removal of the uterus. Typically, to gain access into the abdominal cavity, an incision is made in the abdominal wall and a cannula is passed through the incision to define and maintain an opening through the incision to facilitate passage of surgical instruments and/or tissue. Typically, upon resecting the uterus, or some other tissue depending on the type of surgical procedure performed, the tissue is placed within a specimen bag positioned within the abdominal cavity. An open end of the specimen bag is retracted through the incision in the abdominal wall, and a morcellator is inserted into the specimen bag to morcellate and, in most instances, remove the tissue. Once the tissue is removed from the specimen bag, or once the tissue has been sufficiently reduced in size to permit removal of the specimen bag through the incision, the specimen bag is removed from the abdominal cavity through the incision.

During these procedures, the morcellator may sometimes puncture the specimen bag causing the tissue to leak out of the specimen bag. In addition, the cannula used to maintain the opening may be difficult to insert through the incision and may be challenging to remove from the opening.

Accordingly, there is a need for improved surgical instruments for gaining and maintaining access to a body cavity and/or for providing protection to the specimen bag from the morcellator.

### SUMMARY

In one aspect of the present disclosure, a tissue dilator is provided and includes a plurality of segments. Each segment includes an intermediate portion, a first flange, and a second flange. The intermediate portion has a first end portion and a second end portion. The first flange extends laterally from the first end portion, and the second flange extends laterally from the second end portion. The segments are pivotable between a first configuration, in which the second flanges together define a first channel, and a second configuration, in which the intermediate portions together define a second channel, larger in size than the first channel.

In some embodiments, the first channel and the second channel may define a common longitudinal axis.

It is contemplated that the first flange and the second flange of each segment may overlap one another.

It is envisioned that each segment may have a concave outer surface and a convex inner surface.

In some aspects, the intermediate portion of each segment may have a concave inner surface such that the second channel is cylindrical.

In some embodiments, the first flanges may be positioned and configured to move toward one another as the segments pivot from the first configuration to the second configuration. The first flanges may be positioned and configured to move away from one another as the segments pivot from the second configuration to the first configuration. The second flanges may be positioned and configured to move away from one another as the segments pivot from the first configuration to the second configuration. The second flanges may be positioned and configured to move toward one another as the segments pivot from the second configuration to the first configuration.

It is contemplated that the first flange of each segment may have a first side edge and a second side edge. The first side edge of each of the first flanges may be spaced from a respective second side edge of each of the first flanges of an adjacent segment when the segments are in the first configuration. The first side edge of each of the first flanges may be in abutting engagement with the respective second side edge of each of the first flanges of the adjacent segment when the segments are in the second configuration. The second flange of each segment may have a first side edge and a second side edge. The first side edge of each of the second flanges may be in abutting engagement with a respective second side edge of each of the second flanges of an adjacent segment when the segments are in the first configuration.

It is envisioned that a first segment may have a first side edge defining a plurality of male mating features, and a second segment adjacent the first segment may have a second side edge defining a plurality of female mating features configured to selectively interface with the plurality of male mating features. The male mating features of the first side edge may include a plurality of gears, and the plurality of female mating features of the second side edge may include a plurality of indents. The male mating features of the first side edge may include a plurality of lobes, and the plurality of female mating features of the second side edge may include a plurality of annular cutouts.

In some aspects, each segment may have a petal-shape. In some embodiments, each segment may have a width that tapers in a direction from the first flange to the second flange.

It is contemplated that the tissue dilator may further include an annular member having the plurality of segments pivotably coupled thereto. Each segment may have an outer surface having an outwardly extending protrusion. The annular member may extend through the protrusion of each segment such that the segments pivot between the first configuration and the second configuration about a pivot point defined through the protrusion of each segment.

It is envisioned that in the second configuration, the first flanges and the intermediate portions may define a bell-shaped inner surface.

In some aspects, the segments may pivot as one unit between the first and second configurations.

In another aspect of the present disclosure, a method of dilating tissue is provided. The method includes inserting a tissue dilator in a first configuration into an opening in tissue. The tissue dilator in the first configuration defines a first channel having a first size. The method further includes rotating each segment of a plurality of segments of the tissue dilator toward one another to transition the tissue dilator to a second configuration defining a second channel having a second size greater than the first size. Transitioning the tissue dilator to the second configuration applies pressure on the tissue with each segment to increase a size of the opening in the tissue.

In some embodiments, the method may further include moving the tissue dilator further into the opening in the tissue until a plurality of projections that extend outwardly from the segments engage a bottom portion of the tissue to selectively fix the tissue dilator in the second configuration.

As used herein, the terms parallel and perpendicular are understood to include relative configurations that are substantially parallel and substantially perpendicular up to about +or −10 degrees from true parallel and true perpendicular.

### BRIEF DESCRIPTION OF THE DRAWINGS

The accompanying drawings, which are incorporated in and constitute a part of this specification, illustrate embodiments of the disclosure and, together with a general description of the disclosure given above, and the detailed description of the embodiment(s) given below, serve to explain the principles of the disclosure, wherein:

FIG. 1A is a top, perspective view of an exemplary embodiment of the presently disclosed tissue dilator in a first configuration for insertion into a tissue opening;

FIG. 1B is a bottom, perspective view of the tissue dilator of FIG. 1A in the first configuration;

FIG. 1C is a top view of the tissue dilator of FIG. 1A in the first configuration;

FIG. 2A is a top, perspective view of the tissue dilator of FIG. 1A in a second configuration for dilating a tissue opening;

FIG. 2B is a top view of the tissue dilator of FIG. 1A in the second configuration;

FIG. 3A is a perspective view of an inner surface of a segment of the tissue dilator;

FIG. 3B is a perspective view of an outer surface of the segment of FIG. 3A;

FIG. 4 illustrates a plurality of side views of the tissue dilator of FIG. 1A transitioning from the first configuration to the second configuration;

FIG. 5 is a cross-section, taken along line 5-5, of the tissue dilator of FIG. 1C inserted into a tissue opening;

FIG. 6 is a cross-section, taken along line 6-6, of the tissue dilator of FIG. 2B within the tissue opening;

FIG. 7A is a top, perspective view of another embodiment of a tissue dilator in a first configuration for insertion into a tissue opening;

FIG. 7B is a bottom, perspective view of the tissue dilator of FIG. 7A;

FIG. 8A is a bottom, perspective view of another embodiment of a tissue dilator in a first configuration for insertion into a tissue opening;

FIG. 8B is a side, perspective view of the tissue dilator of FIG. 8A in a second configuration for dilating a tissue opening;

FIG. 9 is a perspective view of an inner surface of a segment of the tissue dilator of FIG. 8A;

FIG. 10A is a bottom, perspective view of another embodiment of a tissue dilator in a first configuration for insertion into a tissue opening; and

FIG. 10B is a top, perspective view of the tissue dilator of FIG. 10A in a second configuration for dilating a tissue opening.

### DETAILED DESCRIPTION

Embodiments of the present disclosure will now be described in detail with reference to the drawings, in which like reference numerals designate identical or corresponding elements in each of the several views. As used herein, the term distal refers to that portion of the tissue dilator or associated apparatus which is farthest from the user, while the term proximal refers to that portion of the tissue dilator or associated apparatus which is closest to the user. In the following description, well-known functions or constructions are not described in detail to avoid obscuring the present disclosure in unnecessary detail.

As used herein with reference to the present disclosure, the terms laparoscopic and endoscopic are interchangeable and refer to instruments having a relatively narrow operating portion for insertion into a cannula or a small incision in the skin. Laparoscopic and endoscopic also refer to minimally invasive surgical procedures. It is believed that the present disclosure may find use in any procedure where access to the interior of the body is limited to one or more relatively small incisions, with or without the use of a cannula or other access port, as in minimally invasive procedures.

The aspects of the present disclosure may be modified for use with various methods for retrieving tissue during minimally invasive procedures. Although the embodiments of the present disclosure will be described with reference to a hysterectomy, e.g., uterus removal, the embodiments of the present disclosure may be used or modified for use with other minimally invasive procedures, e.g., cholecystectomy, appendectomies, nephrectomies, colectomy, splenectomy. Unless otherwise noted, the specimen bags of the present disclosure are formed of rip stop nylon or other suitable material. The specimen bags of the present disclosure may be closed using a drawstring or in any other suitable manner, and may include any feature necessary for deploying and/or retrieving the specimen bag from within a body cavity.

As used herein, the term morcellator refers to a surgical instrument for cutting, mincing up, liquefying, or morcellating, tissue into smaller pieces. Morcellators may be powered or hand-operated, and are generally configured to extract the tissue from the specimen bag, via, e.g., a vacuum tube or through the operation of the cutting mechanism, as the tissue is morcellated.

The tissue dilator, morcellator, and the specimen bag and various other instruments, including, but limited to, trocars, cannulas, access ports, and graspers, form systems for gaining access to a body cavity and for removing tissue from the body cavity during minimally invasive surgery. It is envisioned that the tissue dilators of the present disclosure may be modified for use with various instruments. It is further envisioned that the methods of using the tissue dilators of the present disclosure may be modified to accommodate needs of a given procedure and/or the preferences of the surgeon. It is further envisioned that the embodiments disclosed herein may be used to remove any tissue or object from the body.

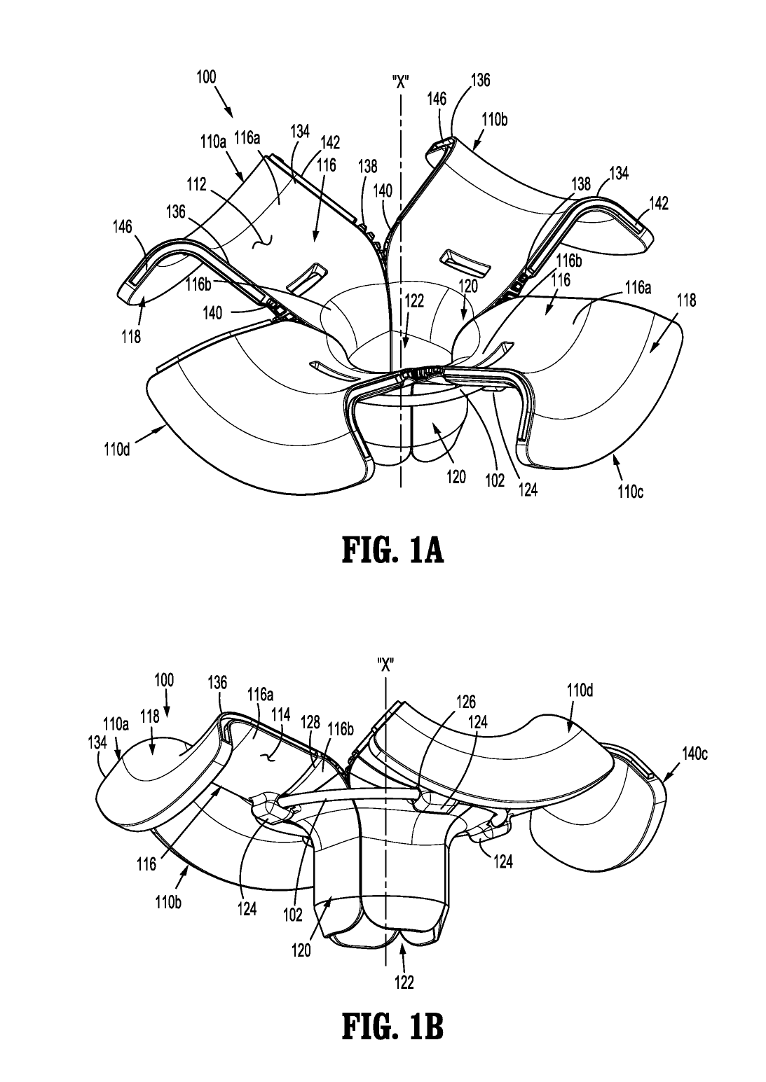

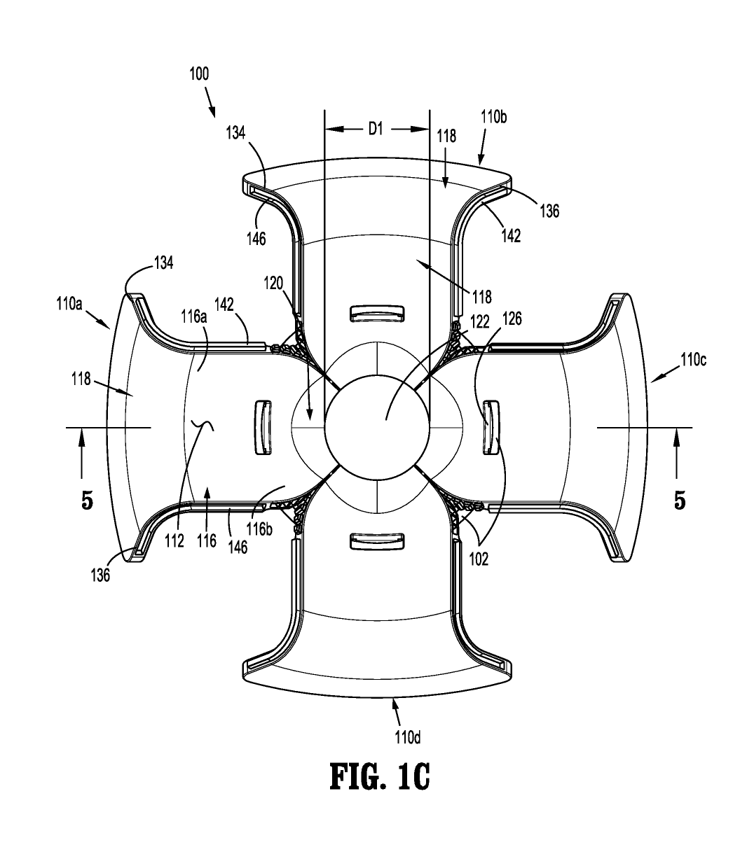

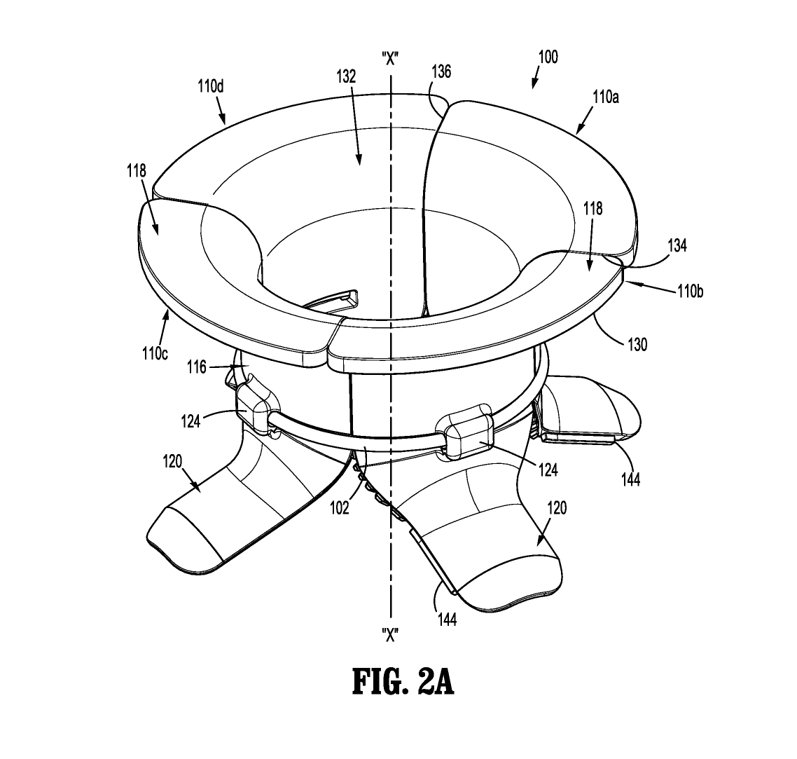

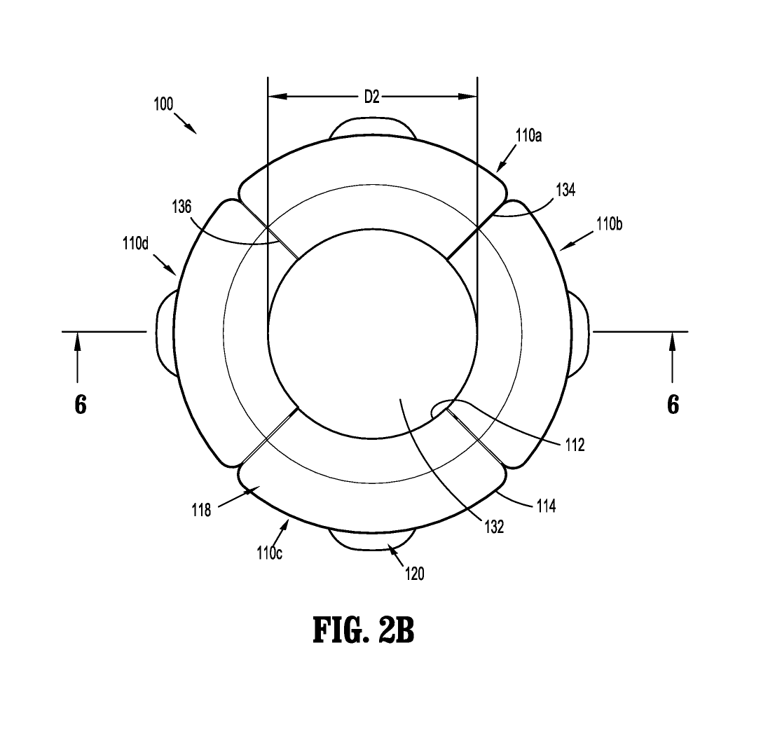

With reference to FIGS. 1A-4, a tissue dilator 100 is configured for insertion into a tissue opening, for example, an incision made in tissue, and to dilate or increase the size of the opening while positioned in the opening. As will be described in detail herein, the tissue dilator 100 is transitionable between a first configuration, shown in FIGS. 1A-1C, in which the tissue dilator 100 defines a first reduced diameter and is configured to be inserted into a tissue opening, and a second configuration, shown in FIGS. 2A and 2B, in which the tissue dilator 100 defines a second enlarged diameter and is configured to increase and maintain the size of the opening.

The tissue dilator 100 generally includes a coupling member, such as, for example, an annular member 102, and four segments 110a, 110b, 110c, 110d pivotally coupled to the annular member 102. In embodiments, the annular member 102 is a loop that defines a central axis “X” therethrough and acts as a support on which segments 110a-d pivot. In some embodiments, the annular member 100 may be replaced with an alternate mechanism for pivotally supporting the segments 110a-d, for example, hinges, bars, ball and sockets, etc. In some embodiments, the tissue dilator 100 may include more or less than four segments.

In embodiments, each segment 110a-d has a generally convex inner surface 112 defined along its length, a generally concave outer surface 114 defined along its length, and a tapering width, thus giving each segment 110a-d a petal-shape. Alternately, other configurations are envisioned. Segments 110a-d generally include an intermediate portion 116, a first flange 118, and a second flange 120. The inner surface 112 of each of the intermediate portions 116 faces the central axis “X” defined through the annular member 100, and the outer surface 114 of each of the intermediate portions 116 faces away from the central axis “X.” The inner surface 112 of each of the intermediate portions 116 is concave between opposing side edges 134, 136 of the intermediate portion 116, and the outer surface 114 of each of the intermediate portions 116 is convex between the opposing side edges 134, 136. As such, when the intermediate portions 116 of each segment 110a-d are in abutting engagement, as shown in FIG. 2A, the inner surface 112 of each of the intermediate portions 116 together define a cylindrical channel 122 (FIG. 1C).

Each of the intermediate portions 116 has a protrusion or block 124 that extends outwardly from the outer surface 114 of each intermediate portion 116. The protrusions 124 are configured to resist backing out of the tissue dilator 100 from a tissue opening while in the second configuration, as will be described in detail below. Each protrusion 124 has a rectangular configuration, but it is contemplated that each protrusion 124 may assume a variety of shapes, such as, for example, triangular, circular, convex, or the like. Each protrusion 124 is circumferentially spaced from an adjacent protrusion 124 by about 90 degrees. It is contemplated that each of the segments 110a-d may include more than one protrusion 124 or that only two of the segments 110a-d have a protrusion 124.

Each protrusion 124 defines a curved bore 126 for rotatable receipt of the annular member 102 of the tissue dilator 100. The bore 126 of each of the protrusions 124 is configured and dimensioned such that the segments 110a-d are pivotable in relation to the annular member 102 about a pivot axis defined by each bore 126. In some embodiments, the annular member 102 may extend through alternate portions of the segments 110a-d, for example, an interior of each of the intermediate portions 116 or through the first or second flanges 118, 120 of the segments 110a-d. The outer surface 114 of each of the intermediate portions 116 defines an elongated cutout 128 therein that is in alignment with the bores 126. The elongated cutout 128 of each of the intermediate portions 116 is configured for receipt of the annular member 102 when the tissue dilator 100 is in the second configuration, as shown in FIG. 2B, to assist in locking the tissue dilator 100 in the second configuration.

With continued reference to FIGS. 1A-4, the intermediate portion 116 of each of the segments 110a-d includes a first end portion 116a, and a second end portion 116b. In embodiments, the first end portion 116a has a substantially uniform width, and the second end portion 116b tapers in width. The first flange 118 of each of the segments 110a-d extends laterally from the first end portion 116a of the intermediate portion 116, and the second flange 120 of each of the segments 110a-d extends laterally from the second end portion 116b of the intermediate portion 116. In embodiments, the first and second flanges 118, 120 are substantially perpendicular relative to the intermediate portion 116 such that first and second flanges 118, 120 are parallel to one another and partially overlap one another. In some embodiments, the first and second flanges 118, 120 may extend at alternate angles relative to the intermediate portion 116, for example, at acute or obtuse angles.

The first flange 118 of each of the segments 110a-d is shaped as a truncated, annular sector that extends outwardly from the first end portion 116a of the intermediate portion 116 and has a greater radius of curvature at its outer end than at the point at which the first flange 118 connects to the first end portion 116a of the intermediate portion 116. As such, the first flanges 118 define a circular flange that extends about the intermediate portions 116 in the second configuration to define an abutment surface as described in detail below. The second flange 120 is also shaped as a truncated, annular sector and extends outwardly from the second end portion 116b of the intermediate portion 116. In contrast to the first flange 118, the second flange 120 of each of the segments 110a-d has a smaller radius of curvature at its outer end than at the point at which the second flange 120 connects to the second end portion 116b of the intermediate portion 116. As such, the second flanges define a tubular body having a diameter that increases in a direction away from the intermediate portion 116 in the first configuration to facilitate entry of the second flanges 120 into an incision as described in detail below. The first and second flanges 118, 120 are monolithically formed with the intermediate portion 116. In some embodiments, the first and second flanges 118, 120 may be integrally connected to the intermediate portion 116.

When the tissue dilator 100 assumes the first configuration, as shown in FIGS. 1A-1C, the second flange 120 of each of the segments 110a-d are in abutting engagement with one another to define a first channel 122 having a central axis coincident with central axis “X.” More specifically, in the first configuration, the concave inner surface 112 of each of the second flanges 120 cooperatively define the first channel 122 about the central axis “X.” The first channel 122 has a first diameter “D1.” The annular member 102 is positioned about the second flanges 120 and may be formed of a resilient material to retain the tissue dilator 100 in the first configuration.

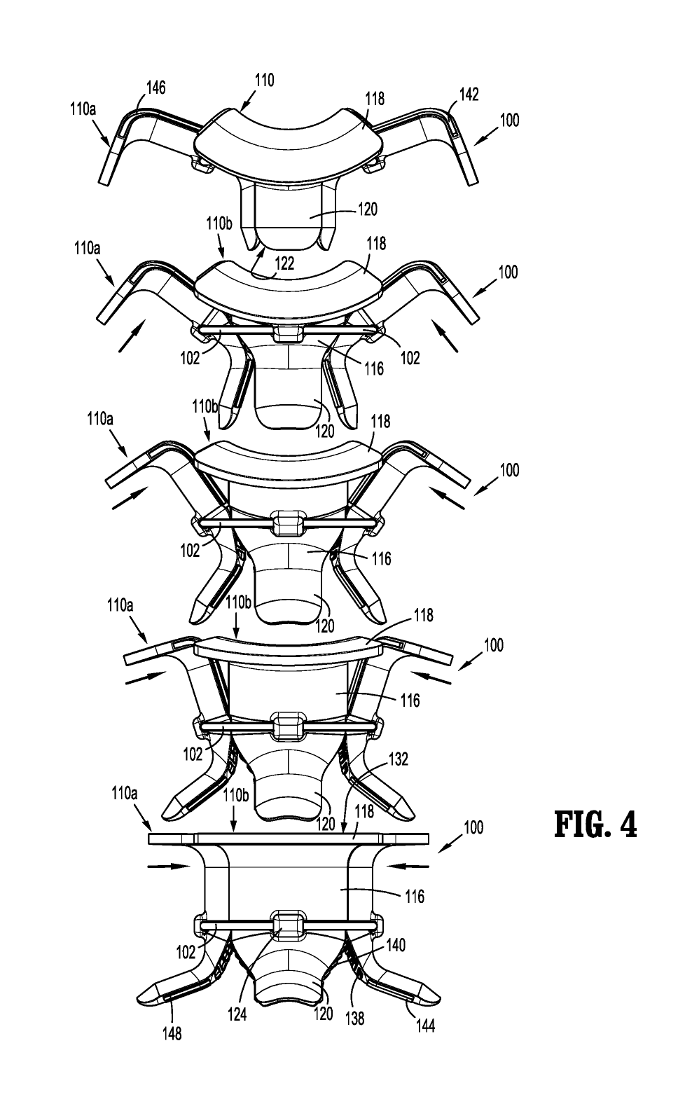

As shown in FIG. 4, as a force is applied to the outer surface 114 of the intermediate portion 116 toward the central axis “X” of the annular member 102 above the annular member 102, the segments 110a-d pivot as one unit toward the second configuration. As the tissue dilator 100 transitions from the first configuration to the second configuration, the first flange 118 of each of the segments 110a-d moves toward one another and the central axis “X” of the annular member 102, and the second flange 120 of each of the segments 110a-d moves away from one another and the central axis “X” of the annular member 102. When the tissue dilator 100 assumes the second configuration, as shown in FIGS. 2A and 2B, the first end portion 116a of each of the intermediate portions 116 are in abutting engagement with one another and the first flange 118 of each of the segments 110a-d are in abutting engagement with one another. The first flange 118 of each of the segments 110a-d extends perpendicularly relative to the central axis “X” of the annular member 102 to define a bell-shaped configuration with each of the segments 110a-d (see FIG. 2A). The first flanges 118 form a circumferential lip 130 configured to be seated on a perimeter of an incision in tissue, as will be described in detail below.

As shown in FIG. 2B, in the second configuration, the intermediate portion 116 of each of the segments 110a-d cooperatively define a second channel 132 having a second diameter “D2,” larger than the first diameter “D1” of the first channel 122. The first and second channels 122, 132 defined by the respective first and second configurations of the tissue dilator 100 define a common central longitudinal axis that is coaxial with the central axis “X” of the annular member 102 due to the symmetrical nature of the segments 110a-d.

With continued reference to FIGS. 1A-4, each segment 110a-d has a first side edge 134 that extends along its length, and an opposing second side edge 136 that extends along its length. The first side edge 134 of each of the segments 110a-d and the second side edge 136 of an adjacent one of the segments 110a-d interface with one another to prevent rotation of any of the segments 110a-d independently of the other segments 110a-d so that each of the segments 110a-d pivot relative to the annular member 102 together as one unit. Although only the first and second adjacent segments 110a, 110b of the four segments 110a-d will be explained in detail below, each of the remaining segments 110c, 110d are identical, and therefore interact with one another in a similar manner as the first and second segments 110a, 110b.

The first side edge 134 of the first segment 110a has a plurality of male mating features, such as, for example, gears 138, and the second side edge 136 of the second segment 110b has a plurality of female mating features, such as, for example, indents 140 configured for receipt of the gears 138. The gears 138 and indents 140 are disposed alongside the second end portion 116b of the intermediate portion 116 of the respective first and second segments 110a, 110b and continue alongside part of the second flange 120 of each of the first and second segments 110a, 110b. The interplay between the gears 138 and the indents 140 maintains axial alignment of the segments 110a, 110b and facilitates pivoting of the first and second segments 110a, 110b with one another as one unit.

The male mating features of the first side edge 134 of the segments 110a-d also include a first elongated projection 142 disposed alongside the first flange 118 and the first end portion 116a of the intermediate portion 116, and a second elongated projection 144 disposed alongside the second flange 120. The female mating features of the second side edge 136 of the segments 110a-d also include a first elongated indent 146 formed alongside the first flange 118 and the first end portion 116a of the intermediate portion 116, and a second elongated indent 148 formed alongside the second flange 120.

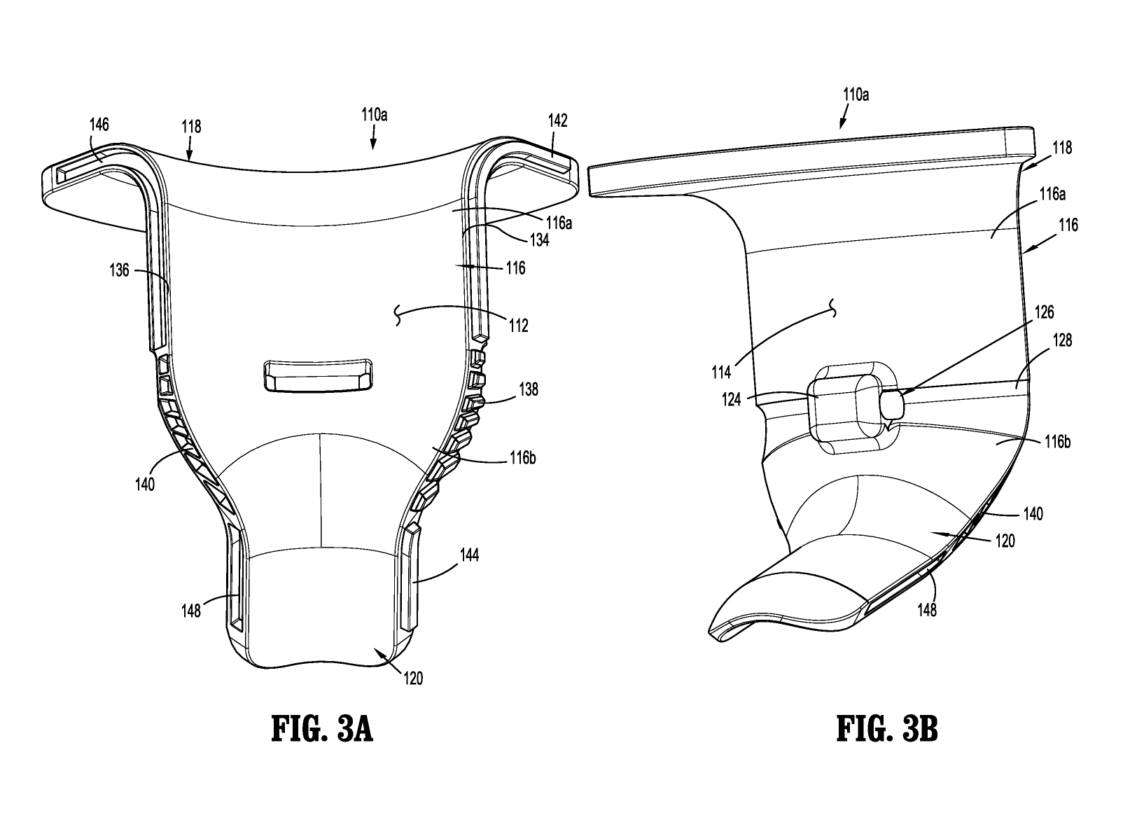

In the first configuration, as shown in FIGS. 1A-1C, the elongated projection 142 of the first flange 118 of the first segment 110a is spaced from the elongated indent 146 of the first flange 118 of the second segment 110b, while the elongated projection 144 (FIGS. 3A and 3B) of the second flange 120 of the first segment 110a is received within the elongated indent 148 (FIGS. 3A and 3B) of the second flange 120 of the second segment 110b. In the second configuration, as shown in FIGS. 2A and 2B, the elongated projection 142 of the first flange 118 of the first segment 110a is received within the elongated indent 146 of the first flange 118 of the second segment 110b, while the elongated projection 144 of the second flange 120 of the first segment 110a is spaced from the elongated indent 148 (FIGS. 3A and 3B) of the second flange 120 of the second segment 110b. The interplay between the elongate projection 142 and the corresponding elongated indent 146 maintains axial alignment of the segments 110a-d when the tissue dilator 100 is in the first configuration, and the interplay between the elongate projection 144 and the corresponding elongated indent 148 maintains axial alignment of the segments 110a-d when the tissue dilator 100 is in the second configuration.

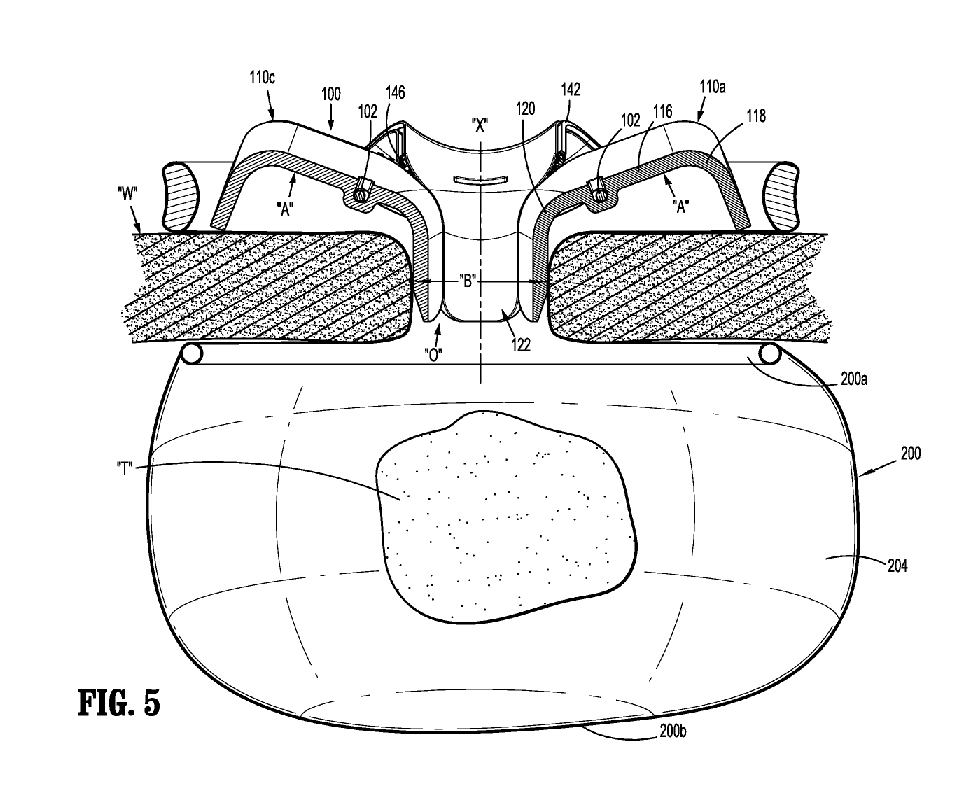

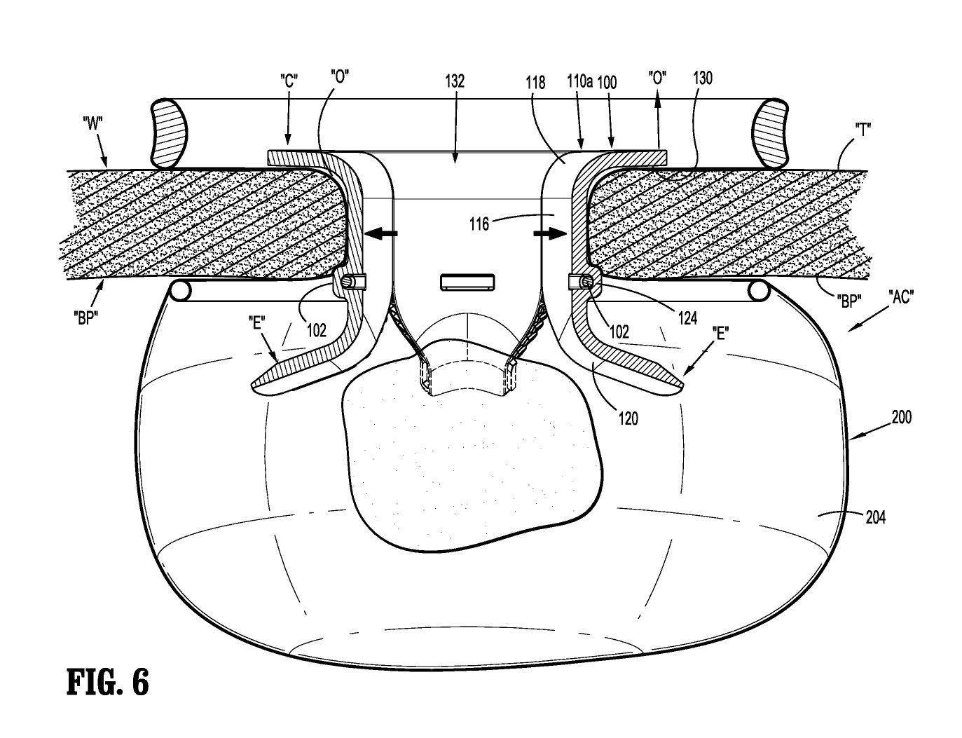

With reference to FIGS. 5 and 6, a surgical system is provided that includes the tissue dilator 100 and a specimen bag 200. The specimen bag 200 includes an open end 200a and a closed end 200b and defines a cavity 204 for receiving tissue to be removed from the patient, e.g., uterine tissue “T.” In some embodiments, the surgical system may include a morcellator (not shown) that is configured to compress and/or break down the resected tissue prior to being removed from the surgical site.

In operation, an incision is made in an abdominal wall “W” of a patient to create an opening “O” in the wall “W.” The tissue dilator 100, while in the first configuration, is inserted within the opening “O” such that the second flange 120 of each of the segments 110a-d enters the opening “O.” In embodiments, the first diameter “D1” of the tissue dilator 100 is between about 0.25 inches and 1 inch such that a surgical instrument of lesser dimensions, e.g., an endoscope, may be passed through the first channel 122 of the tissue dilator 100 to provide confirmation that the tissue dilator 100 is fully engaging the abdominal wall “W.”

A force is applied to at least one of the segments 110a-d at any portion of the segments 110a-d located above the annular member 102 or pivot axis, in the direction indicated by arrow “A” in FIG. 5. Alternately, the force may be applied in an outward direction on any portion of the segments 110a-d located below the annular member 102 or pivot axis. The inwardly-oriented force effects a pivoting of the segments 110a-d from the first configuration toward the second configuration. In particular, the first flange 118 of each of the segments 110a-d move toward one another and the central axis “X” of the annular member 102, and the second flange 120 of each of the segments 110a-d move away from one another and the central axis “X” of the annular member 102. As the segments 110a-d pivot toward the second configuration, the second flange 120 of each of the segments 110a-d applies an outwardly-oriented force, indicated by arrow “B” in FIG. 5, on the inner perimeter of the tissue opening “O,” thereby dilating the opening “O.” The first flanges 118 are prevented from pivoting any further toward one another due to the interface between the elongated projection 142 on each first side edge 134 of segments 110a-d and the corresponding elongated indent 146 on each second side edge 136 of segments 110a-d.

With the tissue dilator 100 moved to the second configuration, a downward force, indicated by arrow “C” in FIG. 6, is applied to the tissue dilator 100 to move the tissue dilator 100 deeper into the abdominal cavity “AC” as shown in FIG. 6. More specifically, the tissue dilator 100 can be pressed into the abdominal cavity “AC” until the annular member 102 and the projection 124 of each of segments 110a-d are disposed below a bottom portion “BP” of the abdominal wall “W.” As such, the abdominal wall “W” is captured in a space defined between the projection 124 of each segment 110a-d and the first flange 118 of each segment 110a-d to prevent an inadvertent transition of the tissue dilator 100 out of the second configuration, and to fix the tissue dilator 100 in the opening “O.”

A morcellator (not shown) may be inserted into the specimen bag 200 through the second channel 132 of the tissue dilator 100 to morcellate the tissue “T.” The morcellator is maintained within the channel 132 of the tissue dilator 100 during the morcellation process. Thus, the tissue dilator 100 acts as a barrier between the morcellator and the specimen bag 200 so that the morcellator does not puncture the specimen bag 200. The specimen bag 200 is then retracted through the second channel 132 of the tissue dilator 100 and out of the abdominal cavity “AC” carrying the tissue “T” therein.

To remove the tissue dilator 100 from the abdominal cavity “AC,” an upward force, indicated by arrow “D” in FIG. 6, is applied to the tissue dilator 100 to dislodge the projection 124 of each of the segments 110a-d from the bottom portion “BP” of the wall “W.” A force is applied to at least one of the segments 110a-d at any portion of the segments 110a-d located below the annular member 102 or pivot axis, in the direction indicated by arrow “E” in FIG. 6. The outwardly-oriented force effects a pivoting of the segments 110a-d from the second configuration toward the first configuration. In particular, the first flange 118 of each of the segments 110a-d move away from one another and the central axis “X” of the annular member 102, and the second flange 120 of each of the segments 110a-d move toward one another and the central axis “X” of the annular member 102. With the tissue dilator 100 assuming the first configuration having the smaller diameter “D1,” the tissue dilator 100 may be removed from the abdominal cavity “AC” through the opening “O.”

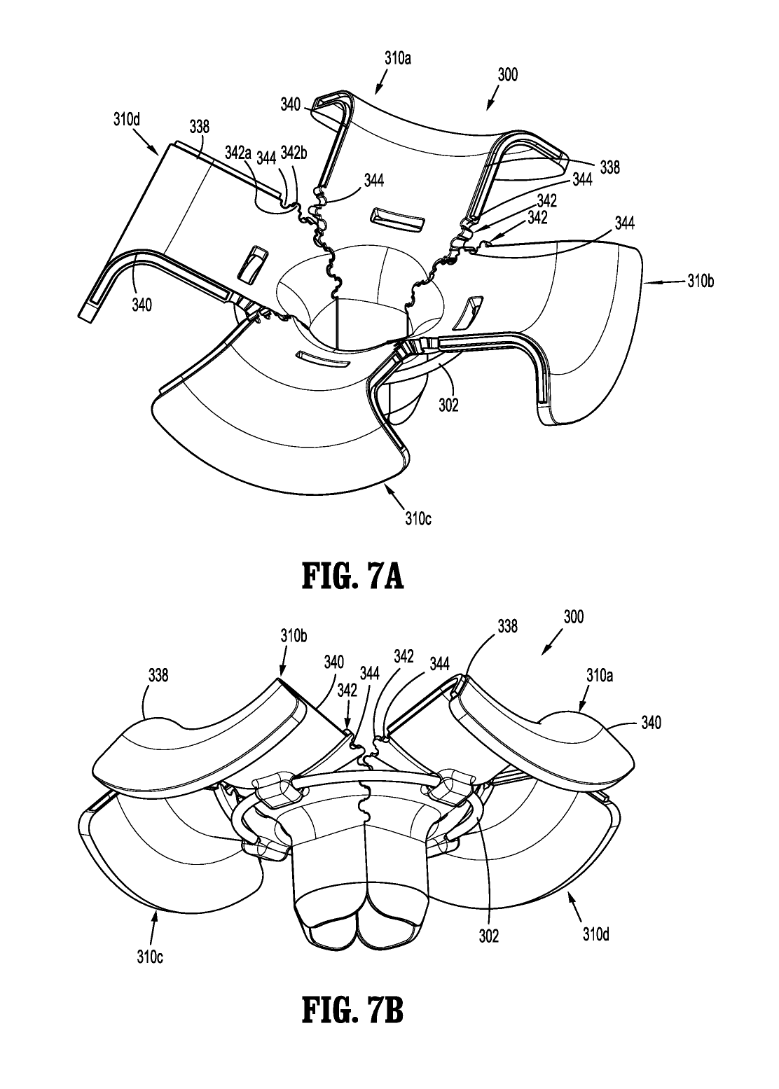

With reference to FIGS. 7A and 7B, another embodiment of a tissue dilator 300 is shown. The tissue dilator 300 of FIGS. 7A and 7B is substantially similar to the tissue dilator 100 of FIGS. 1A-6, and thus will only be described to highlight particular differences between the embodiments.

The tissue dilator 300 includes a plurality of segments 310a, 310b, 310c, 310d pivotably connected to an annular member 302. Each segment 310a-d has a first side edge 338 that extends along its length, and an opposing second side edge 340 that extends along its length. The first side edge 338 of each of the segments 310a-d and the second side edge 340 of an adjacent one of the segments 310a-d interface with one another to prevent rotation of any of the segments 310a-d independently of the other segments 110a-d so that each of the segments 310a-b pivot relative to the annular member 302 together as one unit. Although only the first and second adjacent segments 310a, 310b of the four segments 310a-d are explained in detail below, each of the remaining segments 310c, 310b are identical, and therefore interact with one another in a similar manner as the first and second segments 310a, 310b.

The first and second side edges 338, 340 of the first segment 310a, and the first and second side edges 338, 340 of the second segment 310b each have a plurality of male mating features, such as, for example, lobes 342, and a plurality of female mating features, such as, for example, annular cutouts 334 configured for receipt of the lobes 342. The lobes 342 and the annular cutouts 344 are disposed alongside the first and second side edges 338, 340 of each of the first and second segments 310a, 310b in an alternating pattern. Each lobe 342 has a squared portion 342a and a rounded portion 342b extending outwardly from the squared portion 342a. Each annular cutout 344 has a shape that matches the shape of each lobe 342 such that the first side edge 338 of the first segment 310a and the second side edge 340 of the second segment 310b interlock with one another. The interplay between the lobes 342 and the annular cutouts 344 maintains axial alignment of the segments 310a-d and facilitates pivoting of the first and second segments 310a, 310b with one another as a unit.

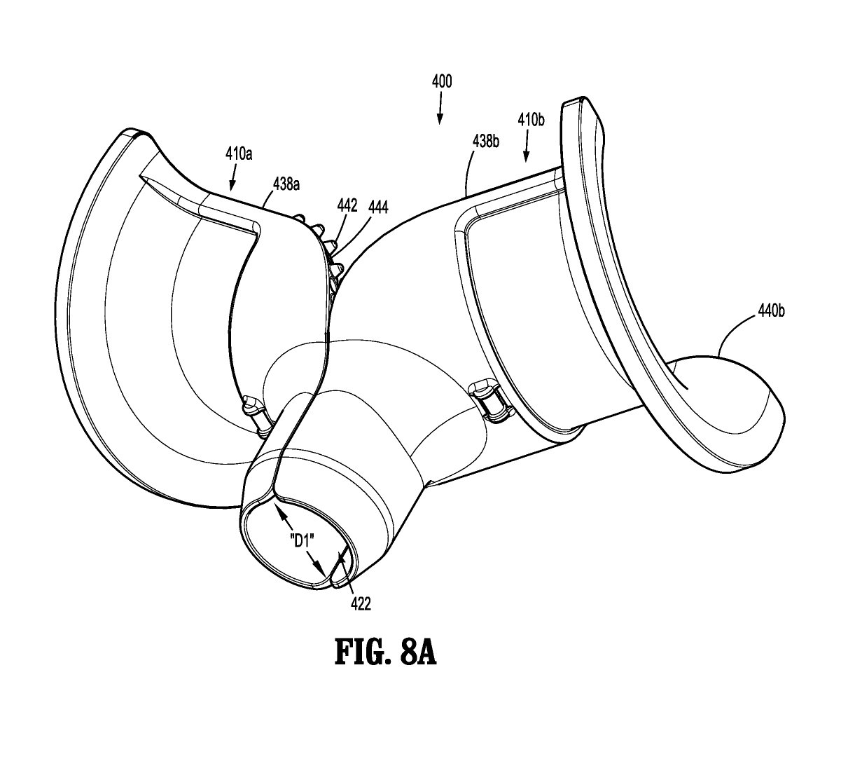

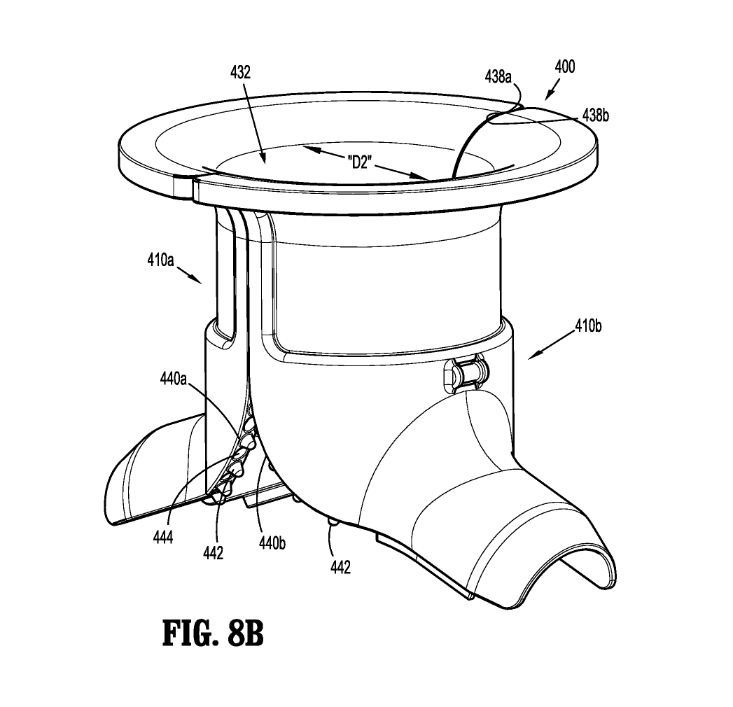

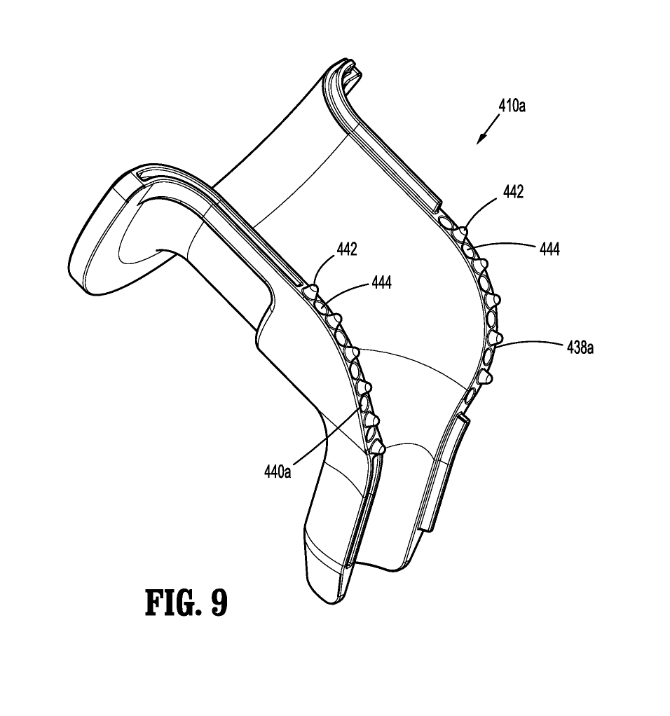

With reference to FIGS. 8A, 8B, and 9, another embodiment of a tissue dilator 400 is shown. The tissue dilator 400 is substantially similar to the tissue dilator 100 of FIGS. 1A-6, and thus will only be described to highlight particular differences between the embodiments.

The tissue dilator 400 includes only two segments 410a, 410b as opposed to the tissue dilator 100 of FIGS. 1A-6, which includes four segments. The two segments 410a, 410b are connected to one another such that the segments 410a, 410b pivot together from a first configuration to a second configuration as one unit. The tissue dilator 400 may include an annular member (not shown), similar to the annular member 102 of tissue dilator 100, pivotally connecting the segments 410a, 410b thereto. The tissue dilator 400 is transitionable between the first configuration, shown in FIG. 8A, in which the tissue dilator 400 defines a first channel 422 having a first diameter “D1” and is configured to be inserted into a tissue opening, and the second configuration, shown in FIG. 8B, in which the tissue dilator 400 defines a second channel 432 having an enlarged, second diameter “D2” and is configured to increase and maintain the size of the opening.

Each of the first and second segments 410a, 410b has a respective first side edge 438a, 438b that extends along its length, and an opposing respective second side edge 440a, 440b that extends along its length. The first and second side edges 438a, 440a of the first segment 410a interface with the first and second side edges 438b, 440b of the second segment 410b, respectively, to prevent rotation of the segments 410a, 410b independently from one another so that the two segments 410a, 410b pivot together as one unit.

The first and second side edges 438a, 440a of the first segment 410a, and the first and second side edges 438b, 440b of the second segment 410b each have a plurality of male mating features, such as, for example, cone-shaped projections or spikes 442, and a plurality of female mating features, such as, for example, cone-shaped holes 444 configured for receipt of the spikes 442. The spikes 442 and the holes 444 are disposed alongside the first and second side edges 438a, 440a and 438b, 440b, respectively, of each of the first and second segments 410a, 410b in an alternating pattern. Each spike 442 has a shape that matches the shape of each hole 444 such that the first and second side edges 438a, 440a of the first segment 410a interlock with the respective first and side edges 438b, 440b of the second segment 410b. The interplay between the spikes 442 and the holes 444 maintains axial alignment of the segments 410a, 410b and facilitates pivoting of the first and second segments 410a, 410b with one another as a unit.

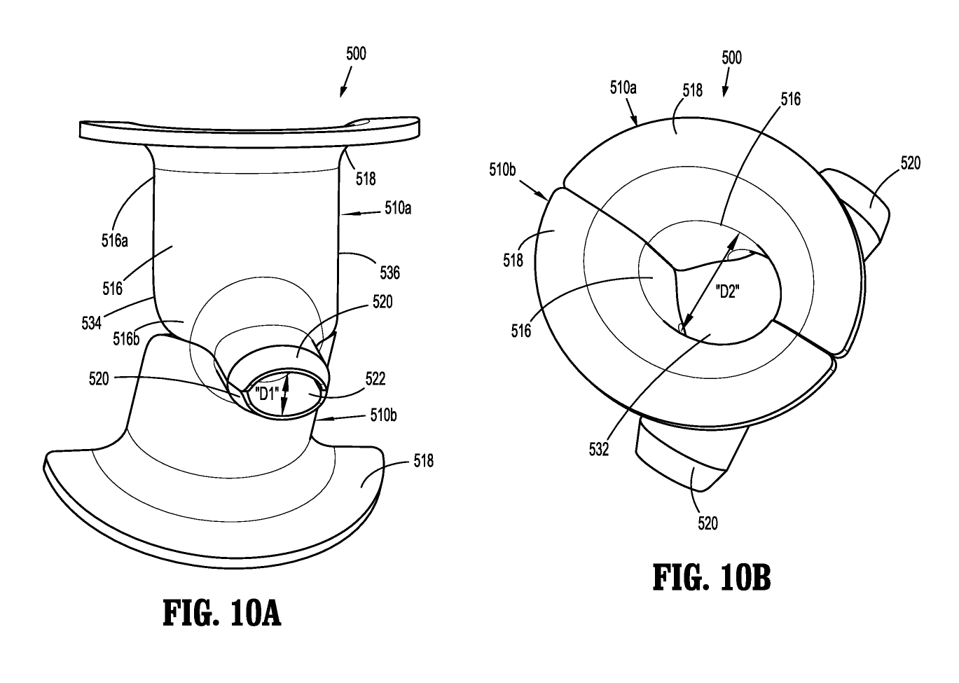

With reference to FIGS. 10A and 10B, another embodiment of a tissue dilator 500 is shown. The tissue dilator 500 is substantially similar to the tissue dilator 100 of FIGS. 1A-6, and thus will only be described to highlight particular differences between the embodiments.

The tissue dilator 500 includes only two segments 510a, 510b as opposed to the tissue dilator 100 of FIGS. 1A-6, which includes four segments. The two segments 510a, 510b are connected to one another such that the segments 510a, 510b pivot together from a first configuration to a second configuration as one unit. The tissue dilator 500 may include an annular member (not shown), similar to the annular member 102 of tissue dilator 100, pivotally connecting the segments 510a, 510b thereto. The tissue dilator 500 is transitionable between the first configuration, shown in FIG. 10A, in which the tissue dilator 500 defines a first channel 522 having a reduced dimension “D1” and is configured to be inserted into a tissue opening, and the second configuration, shown in FIG. 10B, in which the tissue dilator 500 defines a second channel 532 having an enlarged dimension “D2” and is configured to increase and maintain the size of the tissue opening.

Each of the segments 510a, 510b includes an intermediate portion 516, a first flange 518, and a second flange 520. Instead of intermediate portions 516 having the semi-circular shape of the segments of FIGS. 1A-6, the intermediate portions 516 have a half-elliptical shape defined between opposing side edges 534, 536 of the intermediate portion 516. As such, when the intermediate portions 516 of each of the two segments 510a, 510b are in abutting engagement, as shown in FIG. 10B, the intermediate portions 516 together define an elliptically-shaped channel 532. The intermediate portion 516 has a substantially uniform width extending between the first and second end portions 516a, 516b thereof.

The first flange 518 of each of the segments 510a, 510b extends laterally from the first end portion 516a of the intermediate portion 516, and the second flange 520 of each of the segments 510a, 510b extends laterally from the second end portion 516b of the intermediate portion 516. The first flange 518 of each of the segments 510a, 510b is shaped generally as a half-ellipse and extends outwardly from the first end portion 516a of the intermediate portion 516. As such, when the tissue dilator 500 is in the second configuration as shown in FIG. 10B, the first flanges 518 together form an ellipse to define an elliptically-shaped channel 532. The second flange 520 is also shaped as a half-ellipse and extends outwardly from the second end portion 516b of the intermediate portion 516. As such, when the tissue dilator 500 is in the first configuration as shown in FIG. 10A, the second flanges 520 together form an ellipse to define an elliptically-shaped channel 522.

Persons skilled in the art will understand that the devices and methods specifically described herein and illustrated in the accompanying drawings are non-limiting exemplary embodiments. It is envisioned that the elements and features illustrated or described in connection with one exemplary embodiment may be combined with the elements and features of another without departing from the scope of the present disclosure. As well, one skilled in the art will appreciate further features and advantages of the disclosure based on the above-described embodiments. Accordingly, the disclosure is not to be limited by what has been particularly shown and described, except as indicated by the appended claims.

What is claimed is:

1. A tissue dilator, comprising:
a plurality of segments, each segment of the plurality of segments including:
an intermediate portion having a first end portion and a second end portion;
a first flange extending laterally from the first end portion; and
a second flange extending laterally from the second end portion, wherein the plurality of segments is pivotable between a first configuration, in which the second flanges together define a first channel, and a second configuration, in which the intermediate portions together define a second channel, larger in size than the first channel.

2. The tissue dilator of claim 1, wherein the first channel and the second channel define a common longitudinal axis.

3. The tissue dilator of claim 1, wherein the first flange and the second flange of each segment of the plurality of segments overlap one another.

4. The tissue dilator of claim 1, wherein each segment of the plurality of segments has a concave outer surface and a convex inner surface.

5. The tissue dilator of claim 1, wherein the intermediate portion of each segment of the plurality of segments has a concave inner surface such that the second channel is cylindrical.

6. The tissue dilator of claim 1, wherein the first flanges are positioned and configured to move toward one another as the plurality of segments pivot from the first configuration to the second configuration, and the first flanges are positioned and configured to move away from one another as the plurality of segments pivot from the second configuration to the first configuration.

7. The tissue dilator of claim 6, wherein the second flanges are positioned and configured to move away from one another as the plurality of segments pivot from the first configuration to the second configuration, and the second flanges are positioned and configured to move toward one another as the plurality of segments pivot from the second configuration to the first configuration.

8. The tissue dilator of claim 1, wherein the first flange of each segment of the plurality of segments has a first side edge and a second side edge, the first side edge of each of the first flanges being spaced from a respective second side edge of each of the first flanges of an adjacent segment of the plurality of segments when the plurality of segments is in the first configuration.

9. The tissue dilator of claim 8, wherein the first side edge of each of the first flanges is in abutting engagement with the respective second side edge of each of the first flanges of the adjacent segment of the plurality of segments when the plurality of segments is in the second configuration.

10. The tissue dilator of claim 9, wherein the second flange of each segment of the plurality of segments has a first side edge and a second side edge, the first side edge of each of the second flanges being in abutting engagement with a respective second side edge of each of the second flanges of an adjacent segment of the plurality of segments when the plurality of segments is in the first configuration.

11. The tissue dilator of claim 1, wherein a first segment of the plurality of segments has a first side edge defining a plurality of male mating features, and a second segment of the plurality of segments adjacent the first segment has a second side edge defining a plurality of female mating features configured to selectively interface with the plurality of male mating features.

12. The tissue dilator of claim 11, wherein the plurality of male mating features of the first side edge includes a plurality of gears and the plurality of female mating features of the second side edge includes a plurality of indents.

13. The tissue dilator of claim 11, wherein the plurality of male mating features of the first side edge includes a plurality of lobes, and the plurality of female mating features of the second side edge includes a plurality of annular cutouts.

14. The tissue dilator of claim 1, wherein each segment of the plurality of segments has a petal-shape.

15. The tissue dilator of claim 1, wherein each segment of the plurality of segments has a width that tapers in a direction from the first flange to the second flange.

16. The tissue dilator of claim 1, further comprising an annular member having the plurality of segments pivotably coupled thereto.

17. The tissue dilator of claim 16, wherein each segment of the plurality of segments has an outer surface having an outwardly extending protrusion.

18. The tissue dilator of claim 17, wherein the annular member extends through the protrusion of each segment of the plurality of segments such that the plurality of segments pivot between the first configuration and the second configuration about a pivot point defined through the protrusion of each segment of the plurality of segments.

19. The tissue dilator of claim 1, wherein in the second configuration, the first flanges and the intermediate portions define a bell-shaped inner surface.

20. The tissue dilator of claim 1, wherein the plurality of segments pivot as one unit between the first and second configurations.

21. A method of dilating tissue, comprising:
inserting a tissue dilator in a first configuration into an opening in tissue, the tissue dilator in the first configuration defining a first channel having a first size; and
rotating each segment of a plurality of segments of the tissue dilator toward one another to transition the tissue dilator to a second configuration defining a second channel having a second size greater than the first size, thereby applying pressure on the tissue with each segment of the plurality of segments to increase a size of the opening in the tissue.

22. The method of claim 20, further comprising moving the tissue dilator further into the opening in the tissue until a plurality of projections that extend outwardly from the plurality of segments engage a bottom portion of the tissue to selectively fix the tissue dilator in the second configuration.
